(19) United States
(12) Patent Application Publication
(10) Pub. No.: US 2021/0319861 A1
(43) Pub. Date: Oct. 14, 2021

(54) KNOWLEDGE BASE COMPLETION FOR CONSTRUCTING PROBLEM-ORIENTED MEDICAL RECORDS

(71) Applicant: ASAPP, INC., New York, NY (US)

(72) Inventors: James Gustaf Mullenbach, Brooklyn, NY (US); Jordan Louis Swartz, New York, NY (US); Thomas Gregory McKelvey, JR., New York, NY (US); Hui Dai, New York, NY (US); David Sontag, Brookline, MA (US)

(21) Appl. No.: 17/085,927

(22) Filed: Oct. 30, 2020

Related U.S. Application Data
(60) Provisional application No. 63/004,914, filed on Apr. 3, 2020.

(51) Int. Cl. G16H 10/60; G16H 40/20; G16H 50/70; G16H 50/20; G06Q 40/08; G16H 10/40; G16H 20/10; G16H 70/20; G16H 70/40; G06F 40/279; A61B 5/00

(52) U.S. Cl. CPC G16H 10/60; G16H 40/20; G16H 50/70; G16H 50/20; G06Q 40/08; A61B 5/7267; G16H 20/10; G16H 70/20; G16H 70/40; G06F 40/279; G16H 10/40

(57) ABSTRACT

Electronic health records may be organized into problem-oriented medical records. Generating problem-oriented medical record may be based on problem and target relations. Problem and target relations may be determined from a knowledge base. An initial knowledge base may be determined from medical data sets and annotated problem and target relations. The initial knowledge base may be completed to establish new problem target relations using a trained model and/or site embeddings, data statistics, and combined embeddings. The completed knowledge base may be used in generation of problem-oriented medical records.

### CLAIM OF PRIORITY

This patent application claims the benefit of U.S. Patent Application Ser. No. 63/004,914, filed Apr. 3, 2020, and entitled “KNOWLEDGE-BASE COMPLETION FOR CONSTRUCTING PROBLEM-ORIENTED MEDICAL RECORDS” (ASAP-0029-P01).

The content of the foregoing application is hereby incorporated by reference in its entirety for all purposes.

### BACKGROUND

Previously known electronic health record (EHR) systems store patient data chronologically and according to the data type (e.g., medicine, procedure, laboratory results, etc.). Physicians spend a significant portion of their practice time interacting with medical records, which are not organized in a way that promotes efficient analysis.

### BRIEF DESCRIPTION OF THE FIGURES

The invention and the following detailed description of certain embodiments thereof may be understood by reference to the following figures:

FIG. 1 depicts an illustrative detail of a problem from the knowledge base for EHR data for a patient.

FIG. 2 depicts an illustrative example of a data knowledge base relation.

FIG. 3 depicts an illustrative organization of a knowledge base for EHR data for a patient.

FIG. 4 is a schematic diagram of an apparatus to provide a second problem-oriented knowledge base from a first knowledge base.

FIG. 5 is a schematic diagram of an apparatus to generate problem-oriented records.

FIG. 6 is a schematic flow diagram of a procedure to determine a second problem-oriented knowledge base.

FIG. 7 is a schematic diagram of an apparatus to provide a trained model.

FIG. 8 is a schematic flow diagram of a procedure to provide a trained model.

FIG. 9 is a schematic flow diagram of a procedure to construct a vector of features for problem/target pairs.

FIG. 10 is a schematic flow diagram of a procedure to determine an embedding of a problem/target pair.

FIG. 11 is a schematic flow diagram of a procedure to add missing vocabulary to embeddings.

FIG. 12 is a schematic diagram of a system to provide an updated knowledge base.

FIG. 13 is a schematic diagram of illustrative data depicting performance versus problem frequency.

### DETAILED DESCRIPTION

In a variety of applications, physicians and other medical personnel access patient records to make diagnoses and care decisions. Patient records are often organized in chronological order and sometimes organized by data type. Chronological and/or type organization of EHR requires a physician to review and connect disjoint data from various portions of an EHR. For example, many patients who suffer from a disease may take medications to manage the progression of the disease. In order for a physician to determine whether the dose of the medications is adequate from a chronological organization of EHR, the physicians would have to first have to review the problem list of the EHR to see the patient's current medical problems, then scan the medication section to determine what dose the patient is on, and finally navigate to the laboratory section to determine the patient's tolerance to the medication, all of which involves multiple checks and time.

The problem-oriented medical record (POMR) is a paradigm for presenting medical information that, in contrast to chronological presentations, organizes data around the patient's problem list. In the POMR model, all relevant information pertaining to a patient problem may be presented in the same location within the EHR. POMR organization may improve a physicians' ability to reason about each of their patients' problems and save time and errors associated with reviewing chronological EHR.

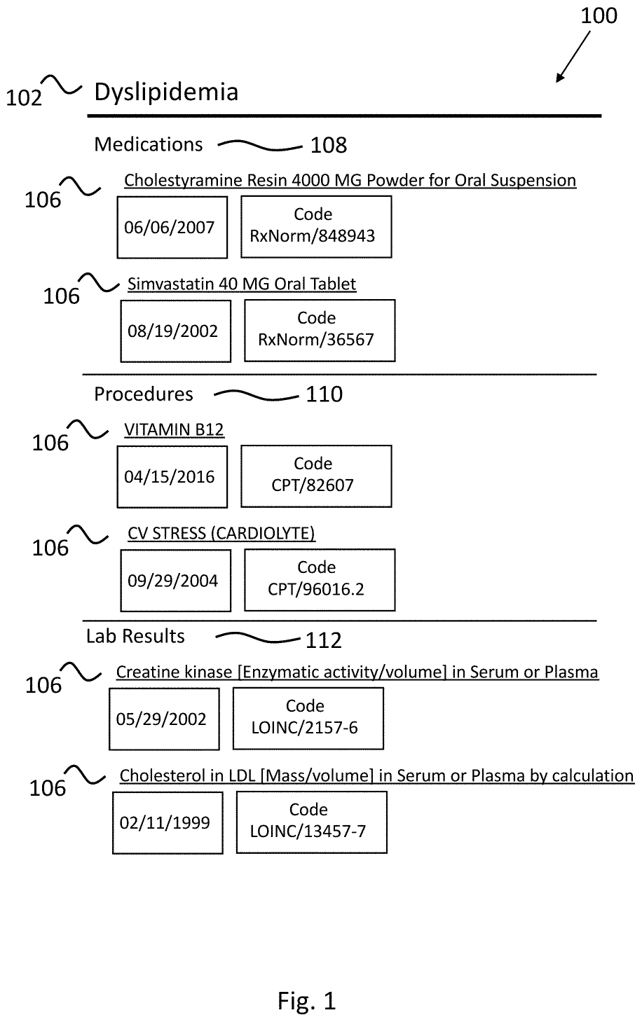

FIG. 1 depicts an illustrative detail 100 of a an example POMR organized around a single identified problem 102. The example detail 100 includes a number of data elements 106 associated with the problem 102 and further organized into a number of record types (e.g., medications 108, procedures 110, and lab results 112). The record types 108, 110, 112 are non-limiting examples, and any record type or other organizing concept are contemplated herein, such as lab results, visit records, comorbidities, or the like. The example depicts medications 108 associated with the problem 102, for example, medications that might be utilized to treat the condition represented by the problem 102. Additionally or alternatively, medications 108 may include one or more medications already prescribed, medications appropriate for prescription, contra-indicated medications, medication cross-sensitivities, and/or the like. Additionally or alternatively, test results, test scheduling, or the like may be associated with data elements 106, allowing operations of the user interface to schedule tests or procedures, to view results, review recommended follow-up activities, and/or the like.

Generation of POMR may include determining problems and linking the problems to their associated labs, medications, procedures, and the like. Traditionally, determining problems and linking the problems to associated data is a manual process that involves multiple experts coming to an agreement while maintaining an adequate level of accuracy. Manual analysis is inefficient and pre-determined associations cannot process and organize problems and associations that were not previously encountered. Additions or changes to problems may require costly and lengthy manual updates to problem lists and associations.

Systems, methods, and apparatuses described herein provide for automatic generation of a knowledge base that may be leveraged for organizing EHR into POMR. The knowledge base may be used to transform a chronological EHR organization into POMR. Systems, methods, and apparatuses described herein automatically link problems to their associated labs, medications, and procedures and are speedier and more flexible than the otherwise manual processes. Systems, methods, and apparatuses described herein use machine learning on electronic health records to automatically construct problem-based groupings of relevant medications, procedures, and/or laboratory tests. Systems, methods, and apparatuses described herein exploit both pre-trained concept embeddings and usage data relating the concepts contained in a longitudinal data set from a large health system.

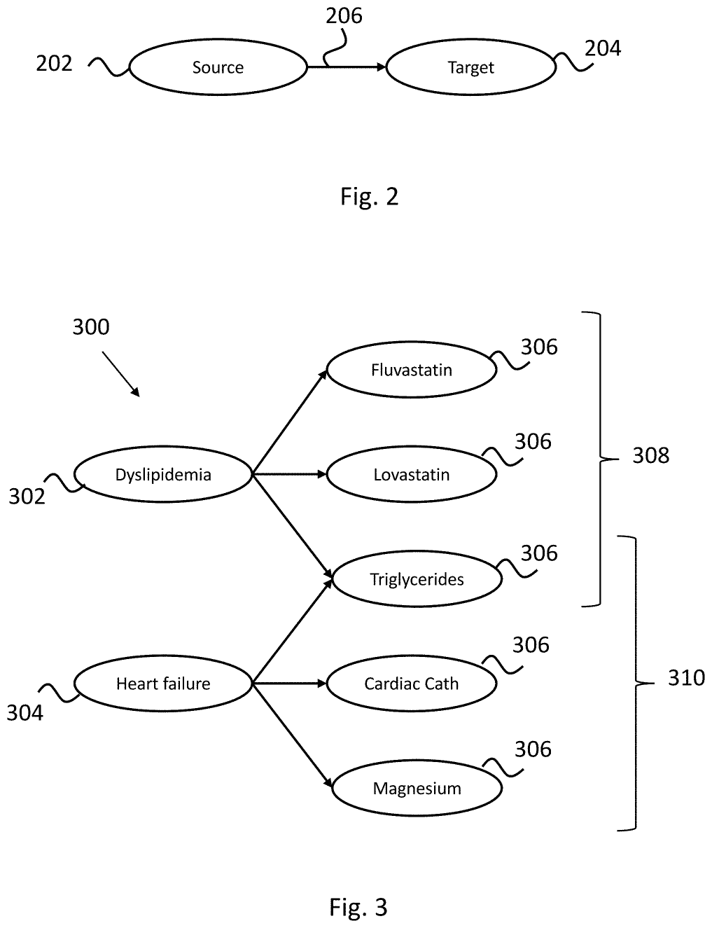

A knowledge base may include a collection of triples that represent a source, target, and a relation between the source and the target. FIG. 2 shows one example of a graph representation of triple. The graph shows a source 202, a target 204, and a relation 206 between the source 202 and the target 204. Triples in a knowledge base may capture associations between problems and targets. A source may be used to represent a problem and a target may be the relevant medications, procedures, laboratory tests, and the like.

FIG. 3 depicts an illustrative organization of a portion of a knowledge base as a graph. The knowledge base graph 200 indicates relations between problems and target entities and may be used to organize EHR into POMR. The example of FIG. 3 depicts identified problems 302, 304 to a number of associated data elements 306, which may be of any data type such as treatments, medicines, lab results, or the like. The knowledge base 300 includes a first set 308 of data elements 306 associated with the first problem 302, and a second set 310 of data elements 306 associated with the second problem 304. The knowledge-base can be represented as lists of medications, procedures, and labs for each problem entity. These lists can then be used downstream as a set of rules to organize patient data around the defined problems. For example, using the knowledge base directly or indirectly (such as by creating a list from the knowledge graph), an algorithm can analyze an EHR to identify diagnosis codes that belong to the problem definition of the knowledge graph and identify all associations to the problem in the EHR. The EHR may then be organized as POMR in a manner such as depicted in FIG. 1.

A knowledge graph may include thousands or even millions of triples. Even in very large graphs, with many nodes and relations, the graph may be incomplete. In some cases, relationships between problems and targets may not be established or defined. New targets may be introduced and may not be related to problems. An incomplete graph may result in incomplete mapping of EHR data to a POMR if some elements in the EHR are not mapped to problems. In embodiments, a knowledge graph may be automatically updated and completed to determine missing relations between problems and targets.

As described herein, the knowledge base may be created automatically. In embodiments, the knowledge base may be updated automatically to define new problems and determine relations between problems and targets. In embodiments, the knowledge base may be updated for specific organizations, medical fields, and the like. The knowledge base may be continuously updated, periodically updated, and/or in response to a trigger such as an indication of new data or an indication from a user. In embodiments, creating and updating the knowledge base may include neural network models that adapt pre-trained medical concept embeddings and learn from both an annotated knowledge-base as well as a longitudinal data set of inpatient and outpatient encounters.

Embedding may be a representation of tokens (i.e., words, phrases, medical concepts) in a vector space such that the embedding includes relevant information about the token. Token embedding may preserve information about the meaning of the token. Two tokens that have similar meanings may have token embeddings that are close to each other in the vector space. By contrast, two tokens that do not have similar meanings may have token embeddings that are not close to each other in the vector space. An embedding may be a vector in an N-dimensional vector space that represents the tokens. For example, the embeddings may be constructed so that tokens with similar meanings or categories are close to one another in the N-dimensional vector space. Embeddings may use larger vector spaces, such as a 128-dimensional vector space or a 512-dimensional vector space.

Any appropriate techniques may be used to compute embeddings. For example, the words may be converted to one-hot vectors where the one-hot vectors are the length of the vocabulary, and the vectors are 1 in an element corresponding to the word and 0 for other elements. The one-hot vectors may then be processed using any appropriate techniques, such as the techniques implemented in Word2Vec or GloVe software. A word embedding may accordingly be created for each word in the vocabulary. An additional embedding may also be added to represent out-of-vocabulary words.

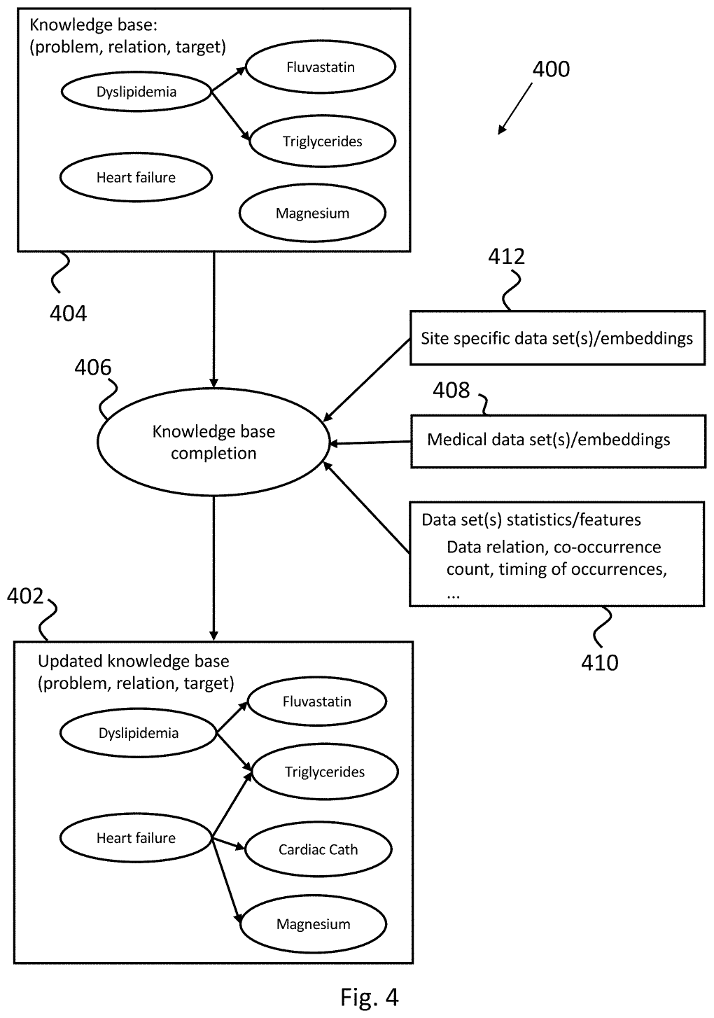

FIG. 4 is a schematic diagram of an apparatus 400 that may be used to generate a second problem-oriented knowledge base 402 from a first knowledge base 404. The second knowledge base 402 may be an updated first knowledge base 404. The example first knowledge base 404 includes a medical data set and annotated data relating problem elements to target elements (e.g., problems associated with treatments, medications, procedures, tests, etc.). The example apparatus 400 includes a knowledge base completion 406 component (e.g., a knowledge base completion engine, circuit, processor, stored computer-executable instructions, and/or other component configured to functionally execute operations to construct the second knowledge base 402). The example knowledge base completion 406 component receives the first knowledge base 404 and generates a second knowledge base 402 that may have updated relations between problems and targets. The knowledge base completion 406 component may include a trained neural network. The knowledge base completion 406 component may be trained on embeddings for data in the first knowledge base and the first knowledge base (such as annotated data in the first knowledge base). The trained knowledge base completion 406 component may process the first knowledge base and determine scores for triples of the first knowledge base, which may be used to identify new targets for problems and/or identify new relations between problems and targets in the first knowledge base.

In some cases, the knowledge base completion 406 component may be further trained on additional medical data sets and external embeddings 408, site-specific data sets and embeddings 412, and/or data set statistics/features 410 such as co-occurrence of data elements in at least a subset of the medical data set 410. The additional data 408, 410, 412 may be used to train the knowledge base completion 406 component to identify missing relations and/or targets in the first knowledge base 404 and generate an updated second knowledge base 402 that includes additional mappings and relations between problems and targets.

In embodiments, the first knowledge base may be an initial knowledge base, such as a seed knowledge base, or may be another knowledge base that was manually or automatically created. The initial knowledge base may be determined from an initial data set. An example medical data set may include a data set including longitudinal health records, inpatient records, outpatient records, and/or emergency department information from an appropriately scoped record set, such as a large regional healthcare system. An example medical data set may include associated diagnoses codes for at least a portion of the records, where the diagnoses codes may correspond to medical problems or may use a separate codification system. The example medical data set may be anonymized, for example, with the removal of names or identifying information, shifting of dates, or the like. An example medical data set may be encounter-based, for example, with each encounter having an associated set of diagnoses codes, medications, procedures, tests, and/or the like.

The initial knowledge base may be generated by determining a problem set. In embodiments, the problem set may be obtained from Clinical Classifications Software (CCS) or diagnosis-related groups (DRG). In embodiments, the problem set may be derived from the medical data set. The problem set may be defined as a set of diagnosis codes from the data set. To define new problems, an annotator (such as an emergency medicine attending physician) may be presented with a list of diagnosis codes ranked by how many unique patients in the data set were associated with the code at any point in their history. In some cases, codes may be limited according to codes that appear in a threshold number of records, such as at least 50 patient records in the data set. The list of codes may be annotated by assigning a diagnosis code to a new problem definition as appropriate. Problem sets may be expanded as needed for an application or field of use by adding or subtracting problems. An example problem set derived from the data set is shown in Table 1.

Referencing Table 1, an example problem set for an implementation is depicted for purposes of illustration. The example problem set will depend upon the initial data set, the relevant problems represented therein, and the problem definition operations, including the utilization of site-specific problems, standardized problems, and/or combinations of these.

TABLE 1
Problem set for an example embodiment

Anemia
Asthma
Arthritis
Atrial fibrillation
Back pain
Cholelithiasis
Chronic kidney disease
Chronic obstructive pulmonary disease
Coronary artery disease
Cough
Dermatitis
Diabetes
Diverticulosis/Diverticulitis
Dyslipidemia
Gastroesophageal reflux disease
Gout
Headache
Heart failure
Hematuria
Hypertension
Hypokalemia
Kidney stone
Mood disorders, including depression
Osteoporosis
Rheumatoid Arthritis
Seizure disorder
Sleep apnea
Syncope
Thrombocytopenia
Thyroid hormone disorders
Uterine fibroid
Urinary tract infection

In embodiments, the determination of an initial knowledge base may include an annotation process for the medical data set. The annotation process may collect a set of annotated triplets (problem, relation, target). In some embodiments, annotated triplets may be determined from the previously generated problem and target lists. In some embodiments, annotated triplets may be obtained from experts by presenting experts with a problem list of candidate medication, laboratory, and procedure codes derived from the data set in relation to each problem from a list. In embodiments, annotations and triplets may be determined from user input and interactions with data from an interface.

In embodiments, annotation may be performed on a subset of triplet candidates. Subset candidates may be determined using an importance score between a problem and an associated data element such as medication, procedure, or lab, for example, as set forth in equation 1:

IMPT=log(p(xᵢ=1|yⱼ=1))−log(p(xᵢ=1|yⱼ=0)) Equation 1.Importance score

In the example of equation 1, xᵢ is a binary variable denoting the existence of a medication, procedure, lab, or other treatment aspect (e.g., follow-up schedules, monitoring, contra-indication, etc.) occurring in an encounter record with a reported diagnosis code, and yⱼ is a binary variable denoted the presence of a diagnosis code in the definition of problem j in an encounter record. The example importance score (IMPT) captures the increase in likelihood of a medication, procedure, lab, etc., appearing in an encounter record when a given problem is also recorded in that record. In embodiments, the top 50 or top 100 codes may be presented for annotation for each problem in a problem list. Annotators may score each problem candidate pair. Scoring may be based on a binary indication of relevance. In one embodiment, relevance may indicate that the candidate would be of interest to a physician. In embodiments, other scoring methods may be used, such as 1-10, continuous range, and the like. Positive and negative relations may be recorded and used in the knowledge base. Negative relations may indicate that a relation between a problem and target should not be present in the knowledge base.

The initial list of candidate codes may be expanded, for example, by performing a second round of annotation using a model trained on the first set. An example operation to expand the initial list of candidate codes replaces the importance score with a relation-specific scoring function (e.g., g(r)) applied to each triplet using a three-way dot product, as depicted in equation 2.

The initial knowledge base comprising annotated triples and medical data set may be processed using the knowledge base completion 406 component that may include a model. A model of the component 406 may be initialized, trained, and used to determine new relations in the initial knowledge graph.

An example operation to initialize and pre-process model (such as a neural network model), which may be a part of, or all of, training the neural network model, includes using external embeddings representing concepts from standardized sets such as RxNorm, Current Procedural Terminology (CPT) and/or Logical Observation Identifiers Names and Codes (LOINC) to initialize parameters for medication, procedure, lab codes, and/or other data elements when the embedding codes are present in the initial data set of the first knowledge base 404. Codes that are not present may be initialized randomly. To initialized problem embeddings, codes in a problem's definition may be combined. An example operation includes translating codes between coding systems (e.g., Systematized Nomenclature of Medicine (SNOMED), International Classification of Diseases (ICD)-10, and/or ICD-9), keeping codes with one-to-one mappings. Embeddings are initialized, in the example, as the weighted average of each definition code, with weights determined according to the frequency of each definition code in the initial data set. An example operation includes initializing relation embeddings to be an all-ones vector, such that the scoring function (e.g., equation 2) reduces to a dot product between the source and target embeddings.

In some cases, external embeddings may have limited efficacy on site-specific codes from an internal vocabulary. External embeddings may have limited efficacy on standardized codes that don't appear in the embeddings' vocabularies. For codes missing from the external vocabulary, embeddings may be randomly initialized. In some cases, initialization of embeddings for missing codes includes acquiring embeddings for them by training on the site data set and exploit nearest neighbors of the codes. Specifically, for each code missing from the external vocabulary, its embedding may be initialized by first finding the k nearest neighbors of the code in the site embedding space, limited to those codes that do exist in the external vocabulary. In some embodiments, k may be configurable to any number and may depend on the size and configuration of the knowledge base and/or data sets. Initialization of the embedding may further take the element-wise average of the corresponding external embeddings of those neighbor codes and use that to initialize an embedding for the missing code.

After initialization, the model (which may include the DistMult model) of the knowledge base completion component 406 may be trained using the ranking loss, which guides the model to rank true triplets higher than randomly sampled negative triplets, with a margin. In certain embodiments, the data set includes explicit negative examples that result from the annotation process. The negative examples may improve the training over random sampling from the vocabulary. Negative examples may be ranked highly according to an importance score, which may improve learning. The training set may be shuffles so that during training, each batch consists of a random selection of positive and negative examples. The model may be optimized with gradient descent. Learning rate and batch size may be tuned by pilot experiments using the validation set.

An example operation to train the model includes the relation-specific scoring function (e.g., g(r)) applied to each triplet of the initial knowledge base 404 using a three-way dot product, as depicted in equation 2:

$$\begin{matrix}Relation \text{-} specific scoring function & \\ {g}_{E M B}^{( r )} = ( {e}_{s} , {e}_{t} ) = {\sum }_{i}^{d} {e}_{{s}_{i}} {e}_{{r}_{i}} {e}_{{t}_{i}} . & Equation 2\end{matrix}$$

In the example of equation 2, eₛ is the source (problem) embedding, eₜ is the target embedding, and d is the dimensionality of embeddings. The example of equation 2 utilizes a DistMult approach. Higher ranked triplets may indicate a true triplet

Additionally or alternatively, an example knowledge base completion 406 component trains and uses its own embeddings (e.g., site-specific embeddings) 412, allowing for greater coverage of the codes used and the possibility of including internal codes without mapping. Example training embeddings on a data set include training a skip-gram model that treats each encounter as a unit, using the entire set of codes in an encounter as context for a given code. In the example, problem embeddings are initialized using an unweighted average of definition code embeddings. Site-specific embeddings may be utilized in addition to and/or as a replacement for scored embeddings described previously.

In certain embodiments, the implementation of a problem-oriented medical record includes a significant aspect that is institution-specific. Accordingly, the neural network model learns from both concept-level information and site-level information. An example knowledge base completion 406 component builds features from the statistics of the data set 410. Example operations to build features from the statistics include counting co-occurrences of each problem/target pair in the data set and normalizing by the count of the target. An example further includes counting each occurrence once per patient. Example operations to determine that a problem/target paid has co-occurred include one or more of: determining that an explicit relation exists between the two in the data (e.g., an annotated diagnostic code corresponding to a problem definition in a record, with the target also listed in the record); determining that the problem/target appear in the same encounter; determining that the problem appears within a time window and at a same facility as the target (e.g., +/−two weeks, three days, and/or other time parameter, which may be symmetric or asymmetric for past/future determinations, and/or according to rules related to the problem and/or target); and/or determining that the problem appears within a time window of the appearance of the target, at any facility (e.g., which may be the same or a distinct time window relative to the time window at the same facility determination).

An example medical data set 408 further includes statistics and/or features 410 for the medical data set 408, for example, including data relations, co-occurrence counts (e.g., for problems, medications, diagnoses, etc.), and/or timing of co-occurrences (e.g., time windows before and/or after a treatment, whereby the occurrence of a problem is considered to be a co-occurrence).

Example operations of the knowledge base completion 406 component include using the vectors of features constructed from the data set, which may further include adjusting the scoring function. For example, referencing equation 3, a similar bilinear term is utilized to combine the specialty feature vectors for problem and target, using a separate set of relational parameters. Referencing equation 4, other features f(s,t) are concatenated with the scores from embeddings and specialty feature vectors to determine a final score. In the example of equation 3, the v values represent the feature vectors, where vᵣ_{ᵢ} are relational parameters, which may be initialized to all (1) ones. In the example of equation 4, the f(s,t) represent engineered features from the data set, and the ⊕ operator represents a concatenation operation.

$$\begin{matrix}Feature vector altered score & \\ {g}_{S P E C}^{( r )} = ( {v}_{s} , {v}_{t} ) = {\sum }_{i}^{d} {v}_{{s}_{i}} {v}_{{r}_{i}} {v}_{{t}_{i}} . & Equation 3 \\ Final score & \\ {g}^{( r )} ( s , t ) = {θ}^{T} [ {g}_{E M B}^{( r )} ( s , t ) ⊕ {g}_{S P E C}^{( r )} ( s , t ) ⊕ f ( s , t ) ] ( {v}_{s} , {v}_{t} ) = {\sum }_{i}^{d} {v}_{{s}_{i}} , {v}_{{r}_{i}} , {v}_{{t}_{i}} . & Equation 4\end{matrix}$$

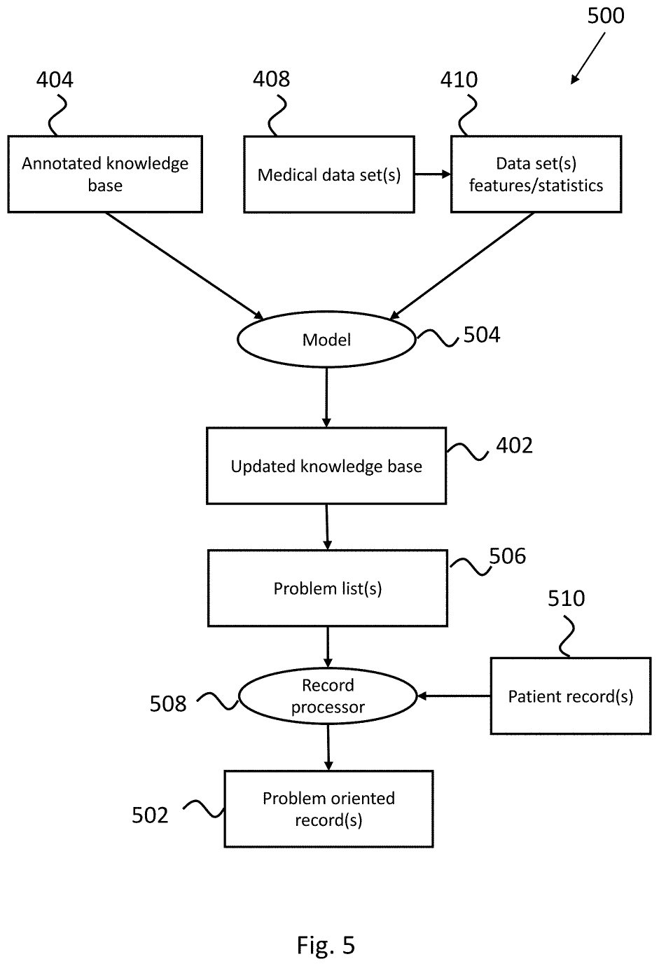

Referencing FIG. 5, an example apparatus 500 to generate problem-oriented records 502 is schematically depicted. The example apparatus 500 includes a modeling component 504 (e.g., a modeling engine, circuit, processor, stored computer-executable instructions, and/or other component configured to functionally execute operations to construct the second knowledge base 402, which may be a part of a knowledge base completion 506 component) that receives a first knowledge base 404, where the first knowledge base includes annotated data relating problems to target elements (e.g., procedures, diagnoses, treatments, test, medications, etc.), and that receives a medical data set 408 (and/or statistics 410 for the medical data set, such as co-occurrences of data). An example first knowledge base 404 includes annotated triples, each including a problem, a target, and a relation between the problem and the target. Example and non-limiting targets include a medication, a procedure, a lab test, a contra-indication, a follow-up description, and/or a monitoring description. The example modeling component 504 trains a neural network model on the first knowledge base and at least a subset of the medical data set 408 (and/or utilizes a trained model, e.g., stored in a memory accessible to the modeling component 504), and scores data relations of data in the first knowledge base with the medical data set 408. The example modeling component 504 constructs a second knowledge base 402 (or updated knowledge base) based on the scored data relations.

The example apparatus 500 includes a problem list 506, for example stored data maintaining a list of problems to be included as organizing concepts for problem-oriented record(s) 502, such as problems defined during a training operation, problems added by a user through a user interface, and/or new problems determined automatically analyzing the second knowledge base 402 and/or the medical data set 408. The example apparatus 500 includes a record processor component 508 that processes the second knowledge base 402 to provide an output, for example, to an insurer, a medical provider, and/or a recipient (e.g., a patient, referral, administrator, relative, etc.). An example record processor component 508 generates a patient problem list (e.g., problems occurring within a patient group, and/or a count or frequency of problems occurring within the patient group) and/or a related medical element list (e.g., treatments, medicines, procedures, etc., as reflected by the problems list). An example record processor component 508 further receives a patient medical record 510 and provides a problem-oriented record 502 (e.g., as a problem-oriented view of the medical record 510). Providing the problem-oriented record 502 includes one or more of storing the problem-oriented record 502 associated with the patient for selective access; providing a visualization, table, or other viewable data element to a user device (e.g., a display screen associated with a medical provider, insurance provider, patient record access, or the like); and/or aggregating the problem-oriented record 502 based on patient medical records 510 for a group of patients.

An example co-occurrence of the data elements (e.g., statistics 410) includes determining a normalized count of co-occurrences among the data elements. An example co-occurrence of the data elements (e.g., statistics 410) includes determining a count of co-occurrences within a time frame represented by data elements in the medical data set 408. In certain embodiments, the example modeling component 504 constructs more than one second knowledge base 402, for example, constructing more than one knowledge base, each having a distinct vocabulary. An example operation to train the neural network model includes training on negative examples in the first knowledge base 404.

An example modeling component 504 determines embeddings from the medical data set 408 and initializes the neural network model based on the embeddings. An example modeling component 504 further determines missing vocabulary in the embeddings, determines neighbors of the missing vocabulary in second embeddings, calculates element-wise average value(s) of the neighbors, and adds missing vocabulary to the embeddings, where the added missing vocabulary is initialized with the calculated average value(s).

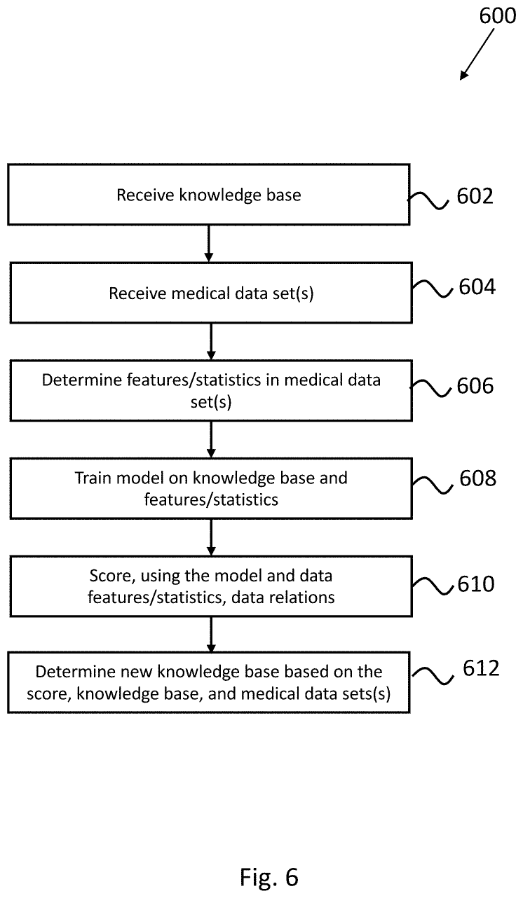

Referencing FIG. 6, an example procedure 600 for determining a second problem-oriented knowledge base is depicted. The example procedure 600 includes an operation 602 to receive a first knowledge base, an operation 604 to receive a medical data set, and an operation 606 to determine features and/or statistics in the medical data set(s). The example procedure 600 includes operation 608 to train a neural network model on the first knowledge base and the determined features and/or statistics in the medical data set(s). The example procedure 600 further includes an operation 610 to score, using the model, the features and/or statistics, and/or relations in the medical data set, and an operation 612 to determine a second updated knowledge base based on the first knowledge base, the score, and the medical data set(s).

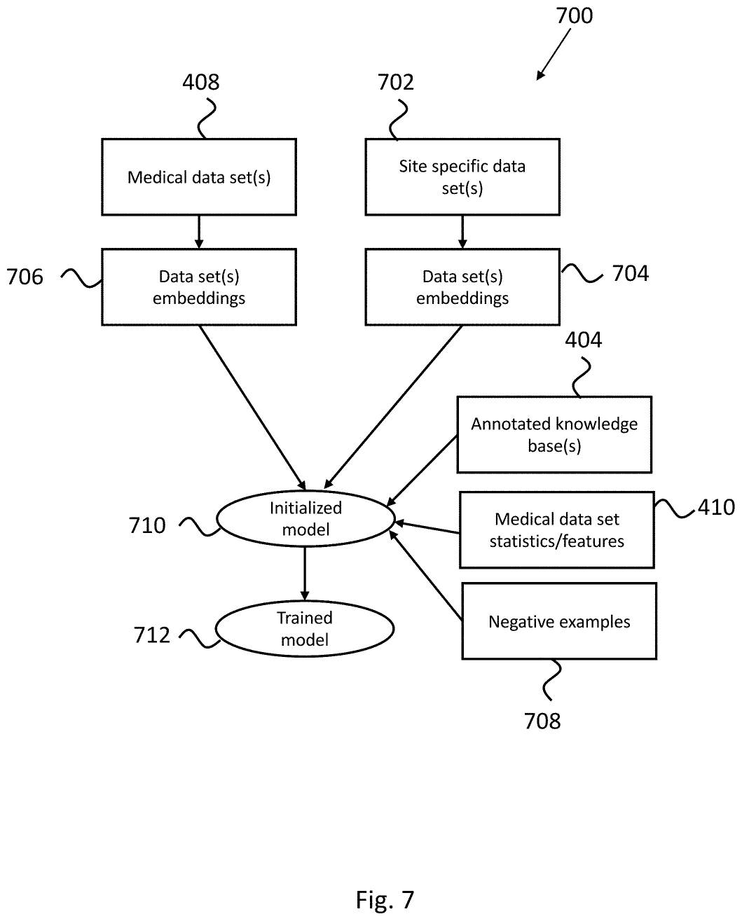

Referencing FIG. 7, an example apparatus 700 for training a model is schematically depicted. Apparatus 700 includes an initialized model 710 component that determines embeddings 706 from the medical data set 408 and initializes a neural network model based on the embeddings. The apparatus 700 further trains the model utilizing an annotated knowledge base 404 and/or features 410 from the medical data set 410. The example apparatus 700 includes a trained model 712 component that provides the trained model for further utilization, such as to determine a POMR, storage of the trained model, and/or display of the trained model and/or parameters of the trained model. An example apparatus 700 further includes a site specific data set 702 and/or embeddings 704 for the site specific data set 702, where the initialized model 710 component further initializes the neural network model with the embeddings 704. An example apparatus 700 further includes negative examples 708 (e.g., which may be included in the annotated knowledge base 404), where the trained model is further trained utilizing the negative examples 708.

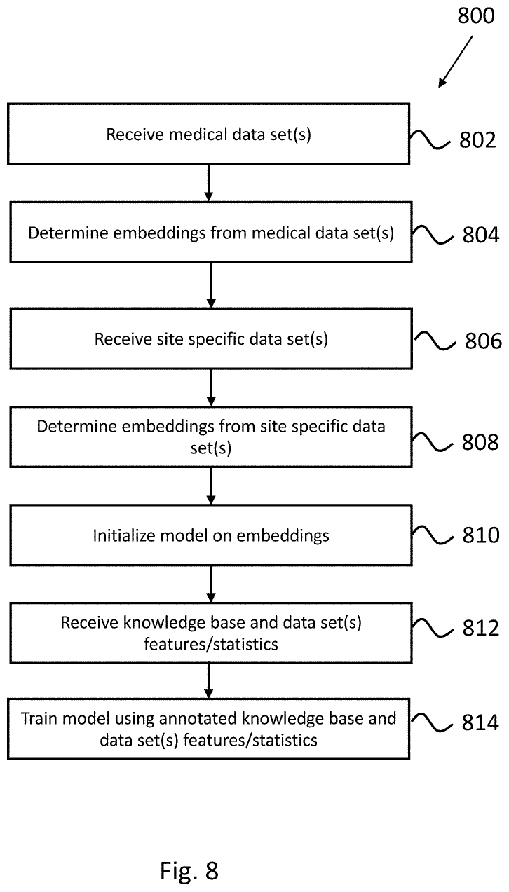

Referencing FIG. 8, an example procedure 800 is schematically depicted for initializing and training a neural network model. The example procedure 800 includes an operation 802 to receive a medical data set, an operation 804 to determine embeddings from the medical data set, an operation 806 (optional) to receive a site specific data set, and an operation 808 to determine embeddings from the site specific data set. The example procedure 800 further includes an operation 810 to initialize the neural network model on the embeddings, an operation 812 to receive a knowledge base (e.g., an annotated knowledge base) and features and/or statistics from the medical data set (and/or the site specific data set), and an operation 814 to train the neural network model using the annotated knowledge base and the data set features and/or statistics.

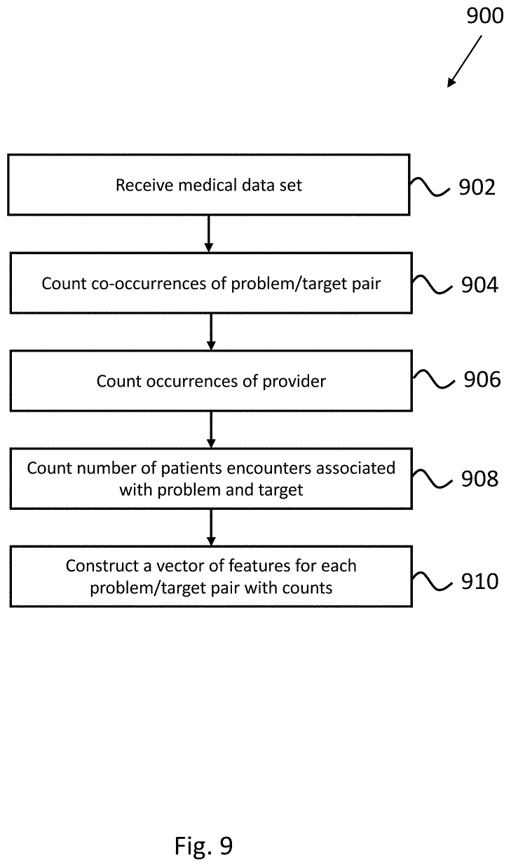

Referencing FIG. 9, an example procedure 900 is schematically depicted for constructing a vector of features, e.g., utilized to train a neural network model as set forth herein. The example procedure 900 includes an operation 902 to receive a medical data set, an operation 904 to count co-occurrences of a problem/target pair (and/or each problem/target pair, or problem/target pairs for a selected set of problems), an operation 906 to count a number of occurrences for a provider, an operation 908 to count a number of patient encounters associated with each problem and target, and an operation 910 to construct a vector of features for each problem/target pair with counts (e.g., counts from operations 904, 906, and/or 908).

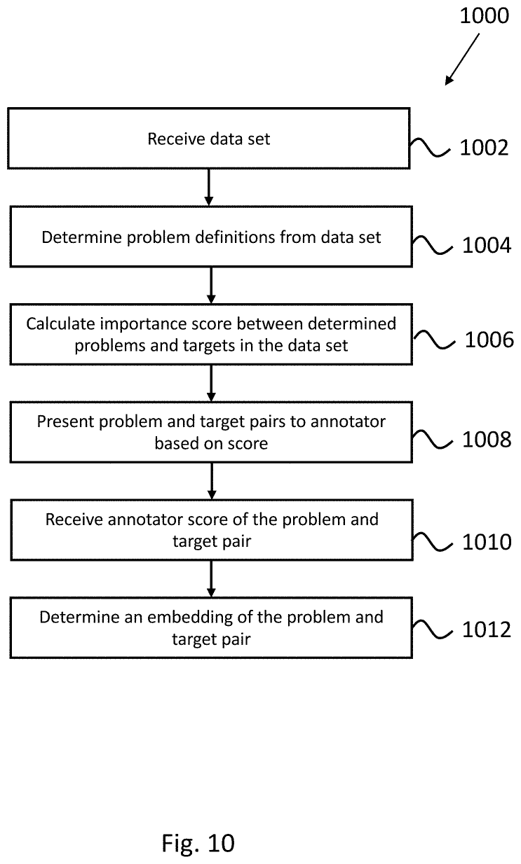

Referencing FIG. 10, an example procedure 1000 is schematically depicted for creating an annotated knowledge base as set forth herein. The example procedure 1000 includes an operation 1002 to receive a medical data set, an operation 1004 to determine problem definitions from the data set, an operation 1006 to calculate importance score between determined problems and targets in the data set, an operation 1008 present problem and target pairs to annotator based on score, an operation 1010 receive annotator score of the problem and target pair, and an operation 1012 Determine an embedding of the problem and target pair.

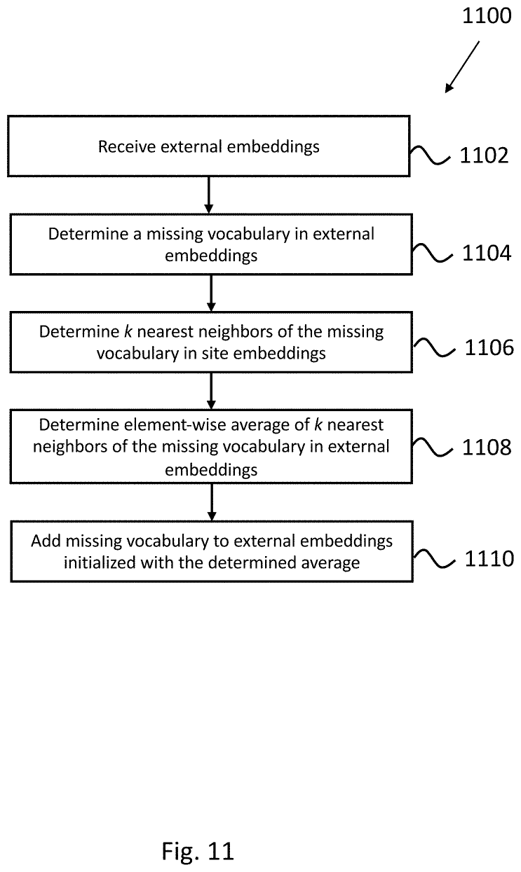

Referencing FIG. 11, an example procedure 1100 to add missing vocabulary elements is schematically depicted. The example procedure 1100 includes an operation 1102 to receive embeddings, an operation 1104 to determine a missing vocabulary element in the embeddings, an operation 1106 to determine k nearest neighbors of the missing vocabulary in site embeddings, and an operation 1108 to determine element-wise average of the k nearest neighbors of the missing vocabulary in the embeddings. The procedure 1100 further includes an operation 1110 to add missing vocabulary to the embeddings, initialized with the determined element-wise average.

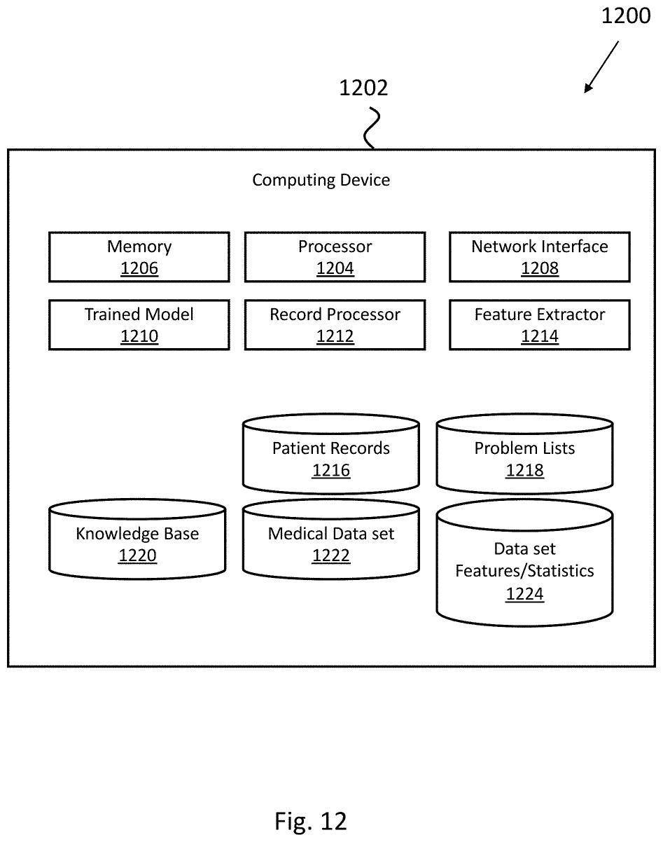

Referencing FIG. 12, an example system 1200 is depicted for determining an updated knowledge base, updating a vocabulary for external embeddings, and/or generating a POMR. The example system 1200 may include, in certain embodiments, any of the systems, apparatus, and/or components set forth throughout the present disclosure. The example system 1200 may be configured to functionally execute any procedures and/or portions thereof as set forth throughout the present disclosure. The example system 1200 depicts elements positioned on a computing device 1202, which may be a single computing device and/or a distributed computing device, with elements positioned on a selected computing device, distributed across more than one computing device, and/or shared between more than one computing device. The example system 1200 schematically depicts a processor 1204 that functionally executes operations of the system 1200, for example, by executing computer-readable instructions stored on a memory 1206. The example system 1200 includes a network interface 1208, for example, executing operations to communicate between computing devices 1202, to access data such as data stored on a cloud server, and/or to interface with user devices such as a health care provider device, an insurance personnel device, a patient device, an annotator device, or the like.

The example system 1200 includes a trained model 1210 component, for example, that stores and/or accesses a trained neural network model that is operated to provide an updated knowledge base and/or determine a POMR. The example system 1200 includes record processor 1212 component that accesses records (e.g., patient records 1216, the medical data set 1222, provider records (not shown), a site-specific data set (not shown), and/or any other records utilized throughout the present disclosure). In certain embodiments, the record processor 1212 controls access and/or permissions to records and/or provides requested records that are selectively processed (e.g., anonymized, time-shifted, or the like). The example system 1200 includes a feature extractor 1214 component configured to extract features as described throughout the present disclosure, including at least in FIGS. 2, 6, and 7, and the related disclosure). The example system 1200 includes a data store of data set features and/or statistics 1224, where the data set features and/or statistics 1224 includes features determined by the feature extractor 1214 and/or records data by which the feature extractor 1214 determines the features. The example system 1200 includes patient records 1216, which may be a part of and/or separate from the medical data set 1222, and which may further include POMR based records. In certain embodiments, the trained model 1210 determines a POMR for a patient based on the patient records 1216, and may store the POMR with the patient records 1216, in a separate data storage, and/or may delete the POMR after usage (e.g., creating a new POMR for each request, for a subset of requests, and/or for certain request types—e.g., creating a new POMR for a patient request, and utilizing a stored POMR for a physician request). The example system includes a data store of a knowledge base 1220, which may be the first knowledge base and/or an annotated knowledge base. In certain embodiments, a second knowledge base determined from the knowledge base 1220 and/or the medical data set 1222, and/or additional knowledge bases created for any purpose (e.g., using a separate problem list 1218, and/or using a distinct vocabulary) may be stored in the same data store with the knowledge base 1220, and/or in a separate data store. The example system 1200 includes a data store with problem list(s) 1218, for example, storing a set of problems of interest for which POMR based records are able to be constructed.

In embodiments, annotated triplets (such as those used to create an initial knowledge base 404) may be split into training, validation, and test sets. An example split includes dividing the annotated triplets at random, with a majority of the annotated triplets forming the training set (e.g., >50%, 70%, etc.) and with the remainder of the annotated triplets divided between validation and test data sets. Example operations include training the neural network model using the ranking loss which guides the model to rank true triplets higher than randomly sampled negative triplets, with a margin. In certain embodiments, the data set includes explicit negative examples that result from the annotation process, which improves the training over random sampling from the vocabulary. The validation set may be utilized to tune the learning rate and batch size with pilot experiments.

In embodiments, rank loss may be computed using any number of functions. One example of determining ranking loss is shown in equation 5, where T is the set of positive annotated examples and T′ is the set of negative annotated examples.

$$\begin{matrix}Ranking loss & \\ L ( Ω ) = {\sum }_{( s , t ) ∈ T} {\sum }_{( {s}^{′} , {t}^{′} ) ∈ {T}^{′}} max \{ {g}^{( r )} ( {s}^{′} , {t}^{′} ) - {g}^{( r )} ( s , t ) + 1 , 0 \} . & Equation 5\end{matrix}$$

Inference includes scoring each triplet (source, relation, target) in the validation set, along with all negative triplets in the validation set having the same problem and relation type. Example metrics computed include the mean ranking (MR) of the true triplet among the set, the mean reciprocal rank (MRR), a first hit frequency (e.g., Hits @ 10, or frequency of the true triplet appearing in the top 10), and/or a second hit frequency (e.g., Hits @ 30, or frequency of the true triplet appearing in the top 30).

Referencing Table 2, example validation data is depicted, providing an illustration of model performance for predicting randomly held-out triplets (e.g., a portion of the medical data set 408 reserved for validation), and/or new data separate from the training data. The table lists externally-trained embeddings and site-specific embeddings trained according to embodiments herein. The table also lists an “Ontology baseline” that combines results from National Drug File Reference Terminology (NDF-RT) and CPT heuristics for medications and procedures (respectively). The set of negatives for the example includes all annotated negatives for a given problem and relation type, but the negatives in the held-out triples are a smaller, random sample of all negative for the problem-relation type pair. Accordingly, results of the trained data set and the held-out triples are not directly comparable. The (Frozen) results are according to the initialized embeddings without any training. It can be seen that the site-specific embeddings lag the externally trained embeddings for the Frozen results. Results are also depicted for relation embeddings, for combined relation and target embeddings, and for combined relation, target, and feature embeddings. The example of Table 2, or similar data for embodiments, provides for evaluation of how performance is developed from aspects of the trained model. In the example of Table 2, the gain from training for site-specific embeddings is larger than the gain for external embeddings. The example of Table 2 suggests that improvements are contributed by the relation parameters and that the addition of engineered features (emphasized in bold on Table 2) provides a strong boost to performance, with comparable performance from the site-specific embeddings relative to the external embeddings.

TABLE 2
Results on illustrative held-out validation data

| | Overall | | Medication | | Procedure | | Labs | |
|---|---|---|---|---|---|---|---|---|
| Model | MR | MRR | MRR | H@5 | MRR | H@5 | MRR | H@5 |
| Ontology | | | 0.07 | 0.02 | 0.121 | 0.246 | | |
| baseline | | | | | | | | |
| External | 9.4 | 0.268 | 0.207 | 0.237 | 0.434 | 0.649 | 0.248 | 0.600 |
| FROZEN | | | | | | | | |
| External | 10.4 | 0.269 | 0.196 | 0.225 | 0.456 | 0.676 | 0.252 | 0.631 |
| RELATION | | | | | | | | |
| External | 9.2 | 0.375 | 0.266 | 0.412 | 0.481 | 0.730 | 0.450 | 0.708 |
| RELATION | | | | | | | | |
| & TARGET | | | | | | | | |
| External | 9.0 | 0.403 | 0.281 | 0.400 | 0.500 | 0.784 | 0.497 | 0.723 |
| External + | 9.2 | 0.461 | 0.353 | 0.525 | 0.442 | 0.676 | 0.605 | 0.754 |
| Features | | | | | | | | |
| Site-specific | 19.7 | 0.170 | 0.188 | 0.188 | 0.233 | 0.216 | 0.113 | 0.092 |
| FROZEN | | | | | | | | |
| Site-specific | 22.6 | 0.196 | 0.190 | 0.212 | 0.251 | 0.270 | 0.170 | 0.215 |
| RELATION | | | | | | | | |
| Site-specific | 15.8 | 0.274 | 0.215 | 0.237 | 0.237 | 0.270 | 0.369 | 0.415 |
| RELATION | | | | | | | | |
| & TARGET | | | | | | | | |
| Site-specific | 14.8 | 0.261 | 0.217 | 0.237 | 0.184 | 0.351 | 0.360 | 0.477 |
| Site- | 10.5 | 0.406 | 0.300 | 0.425 | 0.388 | 0.459 | 0.548 | 0.692 |
| specific + | | | | | | | | |
| Features | | | | | | | | |

Some medical problems are more strongly related with some types of entities. For example, urinary tract infection (UTI) is strongly associated with urinalysis and particular antibiotic medications, but there is not a routinely performed procedure for this common condition. Referencing Table 3, an example performance break-down on the test set is depicted by problem, e.g., to help analyze results in this light. Poor performance in sleep apnea medications may not be important, as there are few medications that directly treat that problem. In designing the POMR, it is not expected that every problem would always have associated labs, medications, and procedures, and performing analyses such as that depicted in Table 3 could be used to decide which suggested elements to turn on.

TABLE 3
Performance by problem

| Problem | Medication | Procedure | Lab |
|---|---|---|---|
| Sleep apnea | 0.00 | 0.67 | 1.00 |
| Hypokalemia | 0.17 | 0.60 | 0.55 |
| Thrombocytopenia | 0.21 | 0.50 | 0.44 |
| Hypertension | 0.60 | 0.80 | 0.91 |
| UTI | 0.82 | 0.80 | 0.97 |

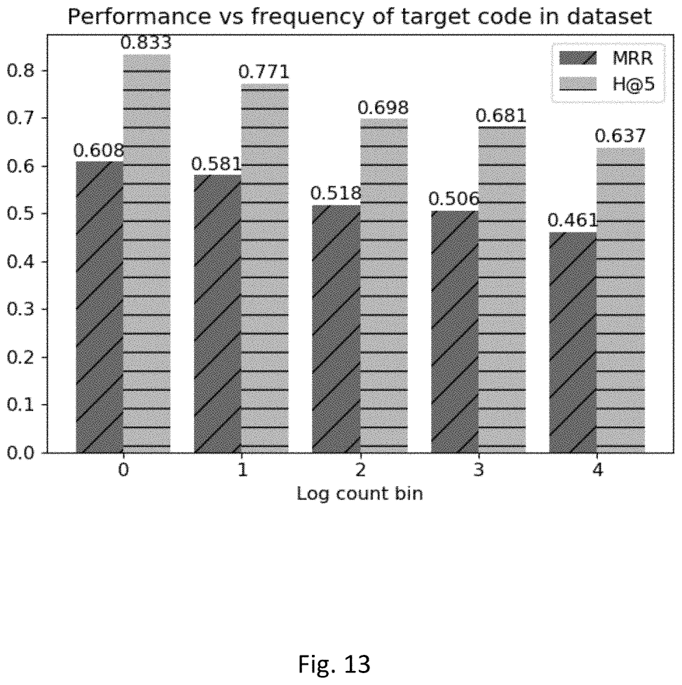

Referencing FIG. 13, an example plot of the performance of the best model (external embedding plus features, in the example) is depicted for different binnings of target code frequency. The example of FIG. 13 utilizes log(X), where X is the count of each target code in the test set, and grouped into bins before computing the metrics. In the example of FIG. 13, codes that are more frequent suffer reduced performance. Further analysis shows that performance is worse than average over target codes that appear in negative examples in the training set. A possible explanation is that during training, the model updates the embeddings for negative target codes to score them lower, but the resulting embeddings end up farther in space from all problem embeddings. More frequent codes are more likely to show up as negative training examples, so performance suffers. It's possible that one could treat all unannotated examples as negatives, and randomly sample them during training in addition to annotated negatives, and that this may mitigate this effect. Example metrics such as those depicted in FIG. 13 may be utilized to determine which features to include and how to treat negative examples in the data set.

Referencing Table 4, an example suggestion set for an implementation is depicted for purposes of illustration. The example suggestion set includes medications, procedures, and/or lab tests according to a trained model, for example, using the 10 highest scoring medications, procedures, and/or lab tests corresponding to the example problem (e.g., “UTI” in the example of Table 1). In the examples of Tables 4-7, the medications, procedures, and labs are depicted separately (e.g., the third-highest suggested medication does not have a specific relationship to the third-highest suggested procedure), and the number of presented suggestions are illustrative, and a different number of presented suggestions may be utilized, and/or the number of suggested medications, procedures, and/or labs may be distinct from each other. The examples of Tables 4-7 depict suggested medication, procedures, and/or labs, but may additionally or alternatively include any other data elements 106, such as follow-up schedules, monitoring schedules, dietary recommendations, and/or any other data elements of interest.

TABLE 4
Example top-10 suggestions from a best-performing model
(medication, procedure, and lab) for problem ″UTI″

| Medication | Procedure | Lab |
|---|---|---|
| Phenazopyridine | Bladder | Piperacillin + tazobactam |
| | lavage/instillation, | [susceptibility] |
| | simple | |
| Ciprofloxacin | US - renal | Bacteria identified in |
| | | isolate by culture |
| Nitrofurantoin | US - abdomen, complete | Ampicillin [susceptibility] |
| Trimethoprim | Abd/pelvis ct w/ + w/o | Bacteria identified in |
| | iv contrast (no po) | unspecified specimen by |
| | | culture |
| Sulfamethoxazole | Cystoscopy/remove | Choriogonadotropin |
| | object, simple | (pregnancy test) |
| | | [presence] in urine |
| Nystatin | Nebulizer treatments | Oxacillin [susceptibility] |
| Cephalexin | Pelvis us, | Nitrofurantoin |
| | transabdominal | [susceptibility] |
| Tamsulosin | Cystoscopy | Aztreonam |
| | | [susceptibility] |
| Ampicillin | Abdomen ct w/o iv and | Penicillin [susceptibility] |
| | with po contrast | |
| Doxazosin | Post void residual bladder | Cefoxitin [susceptibility] |
| | us | |

Referencing Table 5, an example suggestion set for an implementation is depicted for purposes of illustration. The example suggestion set includes medications, procedures, and/or lab tests according to a trained model, for example, using the 10 highest scoring medications, procedures, and/or lab tests corresponding to the example problem (e.g., “Hypokalemia” in the example of Table 1).

TABLE 5
Example top-10 suggestions from a best-performing model
(medication, procedure, and lab) for problem ″Hypokalemia″

| Medication | Procedure | Lab |
|---|---|---|
| Nitroglycerin | (ekg) tracing only | Creatinine [mass/volume] |
| | | in serum or plasma |
| Heparin | Echo, complete (2d), | Lipase [enzymatic |
| | transthoracic | activity/volume) in serum |
| | | or plasma |
| Metoprolol | EKG - hospital based | Glucose [mass/volume) in |
| | | serum or plasma |
| Enoxaparin | Nuc med, myocardial | Natriuretic peptide b |
| | stress pharmacologic | [mass/volume] in serum |
| | | or plasma |
| Lorazepam | EKG complete | Bilirubin.total |
| | (tracing and interp.) | [mass/volume] in serum |
| | | or plasma |
| Labetalol | Chest CT, no contrast | Lymphocytes [/volume] |
| | | in blood by automated |
| | | count |
| Furosemide | Stress echo (exercise) | Sodium [moles/volume] |
| | | in serum or plasma |
| Clopidogrel | Dual-lead pacemaker + | Prothrombin time (pt) |
| | reprogram | |
| Oxygen | Holter hook-up | Leukocytes [/volume] in |
| | (comm prac) | blood by automated count |
| Carvedilol | Echo, stress (dobutamine) | Potassium |
| | | [moles/volume] in serum |
| | | or plasma |

Referencing Table 6, an example suggestion set for an implementation is depicted for purposes of illustration. The example suggestion set includes medications, procedures, and/or lab tests according to a trained model, for example, using the 10 highest scoring medications, procedures, and/or lab tests corresponding to the example problem (e.g., “Thrombocytopenia” in the example of Table 1).

TABLE 6
Example top-10 suggestions from a best-performing model
(medication, procedure, and lab) for problem ″Thrombocytopenia″

| Medication | Procedure | Lab |
|---|---|---|
| Acetaminophen | US - abdomen, complete | Creatinine |
| | | [mass/volume] |
| | | in serum or plasma |
| Prednisone | Filgrastim inj 480 mcg | Reticulocytes/100 |
| | | erythrocytes in blood |
| | | by automated count |
| Metoprolol | Abdomen ct w/o iv and | Hemoglobin |
| | with po contrast | [mass/volume] in blood |
| Cephalexin | Bone marrow biopsy | Glucose [mass/volume] |
| | | in serum or plasma |
| Pegfilgrastim | Bone marrow aspiration | Ferritin [mass/volume] |
| | | in serum or plasma |
| Enalapril | Cxr 2 views ap/pa & | Sodium [moles/volume] |
| | lateral | in serum or plasma |
| Folic acid | Alteplase recomb 1 mg | Lymphocytes [/volume] |
| | | in blood by automated |
| | | count |
| Ibuprofen | Filgrastim inj 300 mcg | Bacteria identified in |
| | | isolate by culture |
| Sulfamethoxazole | Ct - head/brain w/o | Inr in platelet poor |
| | contrast | plasma by coagulation |
| | | assay |
| Glucose | US - abdomen, limited | Circulating tumor cells. |
| | | breast [/volume] in blood |

Referencing Table 7, an example suggestion set for an implementation is depicted for purposes of illustration. The example suggestion set includes medications, procedures, and/or lab tests according to a trained model, for example, using the 10 highest scoring medications, procedures, and/or lab tests corresponding to the example problem (e.g., “Sleep apnea” in the example of Table 1).

TABLE 7
Example top-10 suggestions from a best-performing model
(medication, procedure, and lab) for problem ″Sleep apnea″

| Medication | Procedure | Lab |
|---|---|---|
| Montelukast | Sleep study, w/ cpap | Natriuretic peptide.b |
| | (treatment settings) | prohormone n-terminal |
| | | (mass/volume) in serum |
| | | or plasma |
| Ipratropium | Sleep study, w/o cpap | Creatinine [mass/volume] |
| | | in serum or plasma |
| Fluticasone | Positive airway pressure | Natriuretic peptide b |
| | (cpap) | [mass/volume] in serum |
| | | or plasma |
| Exenatide | (ekg) tracing only | Leukocytes [/volume] in |
| | | blood by automated count |
| Bumetanide | Echo, complete (2d), | Nicotine [mass/volume] in |
| | transthoracic | urine |
| Albuterol | Ekg complete | Inr in platelet poor plasma |
| | (tracing and interp) | by coagulation assay |
| Enoxaparin | Cxr 2 views ap/pa & lateral | Hemoglobin |
| | | [mass/volume] in blood |
| Azithromycin | Pulse ox w/ rest/exercise, | Cotinine [mass/volume] in |
| | multiple (op) | urine |
| Nitroglycerin | Ekg (hospital based) | Glucose [mass/volume] in |
| | | blood by automated test |
| | | strip |
| Glucose | Echo, stress (exercise) | Lymphocytes [/volume] in |
| | | blood by automated count |

The methods and systems described herein may be deployed in part or in whole through a machine that executes computer software, program codes, and/or instructions on a processor. “Processor” as used herein is meant to include at least one processor and unless context clearly indicates otherwise, the plural and the singular should be understood to be interchangeable. Any aspects of the present disclosure may be implemented as a computer-implemented method on the machine, as a system or apparatus as part of or in relation to the machine, or as a computer program product embodied in a computer readable medium executing on one or more of the machines. The processor may be part of a server, client, network infrastructure, mobile computing platform, stationary computing platform, or other computing platform. A processor may be any kind of computational or processing device capable of executing program instructions, codes, binary instructions and the like. The processor may be or include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more thread. The thread may spawn other threads that may have assigned priorities associated with them; the processor may execute these threads based on priority or any other order based on instructions provided in the program code. The processor may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through a machine that executes computer software on a server, client, firewall, gateway, hub, router, or other such computer and/or networking hardware. The software program may be associated with a server that may include a file server, print server, domain server, internet server, intranet server and other variants such as secondary server, host server, distributed server and the like. The server may include one or more of memories, processors, computer readable media, storage media, ports (physical and virtual), communication devices, and interfaces capable of accessing other servers, clients, machines, and devices through a wired or a wireless medium, and the like. The methods, programs, or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers and the like. Additionally, this coupling and/or connection may facilitate remote execution of program across the network. The networking of some or all of these devices may facilitate parallel processing of a program or method at one or more locations without deviating from the scope of the disclosure. In addition, any of the devices attached to the server through an interface may include at least one storage medium capable of storing methods, programs, code and/or instructions. A central repository may provide program instructions to be executed on different devices. In this implementation, the remote repository may act as a storage medium for program code, instructions, and programs.

The software program may be associated with a client that may include a file client, print client, domain client, internet client, intranet client and other variants such as secondary client, host client, distributed client and the like. The client may include one or more of memories, processors, computer readable media, storage media, ports (physical and virtual), communication devices, and interfaces capable of accessing other clients, servers, machines, and devices through a wired or a wireless medium, and the like. The methods, programs, or codes as described herein and elsewhere may be executed by the client. In addition, other devices required for execution of methods as described in this application may be considered as a part of the infrastructure associated with the client.

The client may provide an interface to other devices including, without limitation, servers, other clients, printers, database servers, print servers, file servers, communication servers, distributed servers and the like. Additionally, this coupling and/or connection may facilitate remote execution of program across the network. The networking of some or all of these devices may facilitate parallel processing of a program or method at one or more locations without deviating from the scope of the disclosure. In addition, any of the devices attached to the client through an interface may include at least one storage medium capable of storing methods, programs, applications, code and/or instructions. A central repository may provide program instructions to be executed on different devices. In this implementation, the remote repository may act as a storage medium for program code, instructions, and programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules and/or components as known in the art. The computing and/or non-computing device(s) associated with the network infrastructure may include, apart from other components, a storage medium such as flash memory, buffer, stack, RAM, ROM and the like. The processes, methods, program codes, instructions described herein and elsewhere may be executed by one or more of the network infrastructural elements.

The methods, program codes, and instructions described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like. The cell network may be a GSM, GPRS, 3G, EVDO, mesh, or other networks types.

The methods, programs codes, and instructions described herein and elsewhere may be implemented on or through mobile devices. The mobile devices may include navigation devices, cell phones, mobile phones, mobile personal digital assistants, laptops, palmtops, netbooks, pagers, electronic books readers, music players and the like. These devices may include, apart from other components, a storage medium such as a flash memory, buffer, RAM, ROM and one or more computing devices. The computing devices associated with mobile devices may be enabled to execute program codes, methods, and instructions stored thereon. Alternatively, the mobile devices may be configured to execute instructions in collaboration with other devices. The mobile devices may communicate with base stations interfaced with servers and configured to execute program codes. The mobile devices may communicate on a peer-to-peer network, mesh network, or other communications network. The program code may be stored on the storage medium associated with the server and executed by a computing device embedded within the server. The base station may include a computing device and a storage medium. The storage device may store program codes and instructions executed by the computing devices associated with the base station.

The computer software, program codes, and/or instructions may be stored and/or accessed on machine readable media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g. USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

The methods and systems described herein may transform physical and/or or intangible items from one state to another. The methods and systems described herein may also transform data representing physical and/or intangible items from one state to another.

The elements described and depicted herein, including in flow charts and block diagrams throughout the figures, imply logical boundaries between the elements. However, according to software or hardware engineering practices, the depicted elements and the functions thereof may be implemented on machines through computer executable media having a processor capable of executing program instructions stored thereon as a monolithic software structure, as standalone software modules, or as modules that employ external routines, code, services, and so forth, or any combination of these, and all such implementations may be within the scope of the present disclosure. Examples of such machines may include, but may not be limited to, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices having artificial intelligence, computing devices, networking equipment, servers, routers and the like. Furthermore, the elements depicted in the flow chart and block diagrams or any other logical component may be implemented on a machine capable of executing program instructions. Thus, while the foregoing drawings and descriptions set forth functional aspects of the disclosed systems, no particular arrangement of software for implementing these functional aspects should be inferred from these descriptions unless explicitly stated or otherwise clear from the context. Similarly, it will be appreciated that the various steps identified and described above may be varied, and that the order of steps may be adapted to particular applications of the techniques disclosed herein. All such variations and modifications are intended to fall within the scope of this disclosure. As such, the depiction and/or description of an order for various steps should not be understood to require a particular order of execution for those steps, unless required by a particular application, or explicitly stated or otherwise clear from the context.

The methods and/or processes described above, and steps thereof, may be realized in hardware, software or any combination of hardware and software suitable for a particular application. The hardware may include a general-purpose computer and/or dedicated computing device or specific computing device or particular aspect or component of a specific computing device. The processes may be realized in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine-readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software, or any other machine capable of executing program instructions.

Thus, in one aspect, each method described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or software described above. All such permutations and combinations are intended to fall within the scope of the present disclosure.

While the invention has been disclosed in connection with the preferred embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

All documents referenced herein are hereby incorporated by reference in the entirety.

What is claimed is:

1. A method for completion of a knowledge base for organizing medical records, comprising:
receiving a first knowledge base, the first knowledge base comprising annotated data relating problem elements to target elements;
receiving a medical data set;
determining a co-occurrence of data elements in at least a subset of the medical data set;
training a neural network model on the first knowledge base and the determined co-occurrence of data;
scoring, using the trained neural network model and the determined co-occurrence of the data elements, data relations in the first knowledge base; and
constructing, based on the scored data relations, a second knowledge base.

2. The method of claim 1, further comprising processing the second knowledge base to generate at least one of a medical provider-based output, insurance based output, or a recipient based output.

3. The method of claim 1, further comprising, using the second knowledge base to generate at least one list of patient problems and related medical elements.

4. The method of claim 3, further comprising:
receiving a patient medical record; and
reorganizing the patient medical record into a problem-oriented view using the at least one list.

5. The method of claim 1, wherein determining the co-occurrence of the data elements comprises determining a normalized count of co-occurrences.

6. The method of claim 1, wherein defining the second knowledge base comprises combining two or more knowledge based having different vocabularies.

7. The method of claim 1, wherein training the neural network model comprises training on negative examples in the first knowledge base.

8. The method of claim 1, wherein determining the co-occurrence of the data elements comprises determining a count of occurrences within a time frame represented by the data elements in the medical data set.

9. The method of claim 1, wherein the first knowledge base comprises annotated triples that include a problem, a target, and a relation between the problem and the target.

10. The method of claim 9, wherein the target comprises at least one of a medication, a procedure, or a laboratory results.

11. The method of claim 1, further comprising:
determining embeddings from the medical data set; and
initializing the neural network model based on the embeddings.

12. The method of claim 11, wherein determining embeddings further comprises:
determining missing vocabulary in the embeddings;
determining neighbors of the missing vocabulary the embeddings;
calculating element-wise average value of the neighbors; and
adding the missing vocabulary to the embeddings initialized with the calculated average value.

13. A system, comprising:
at least one server computer comprising at least one processor and at least one memory, the at least one server computer configured to:
receive a first knowledge base, the first knowledge base comprising annotated data relating problem elements to target elements;
receive a medical data set;
determine a co-occurrence of data elements in at least a subset of the medical data set;
train a neural network model on the first knowledge base and the determined co-occurrence of data;
score, using the trained neural network model and the determined co-occurrence of the data elements, data relations in the first knowledge base; and
construct, based on the scored data relations, a second knowledge base.

14. The system of claim 13, wherein the first knowledge base comprises annotated triples that include a problem, a target, and a relation between the problem and the target.

15. The system of claim 13, wherein the at least one server computer is configured to:
determine embeddings from the medical data set; and
initialize the neural network model based on the embeddings.

16. The system of claim 13, wherein the at least one server computer is configured to:
generate at least one list of patient problems and related medical elements using the second knowledge base.

17. The system of claim 16, wherein the at least one server computer is configured to:
receive a patient medical record; and
reorganize the patient medical record into a problem-oriented view using the at least one list.

18. One or more non-transitory, computer-readable media comprising computer-executable instructions that, when executed, cause at least one processor to perform actions comprising:
receiving a first knowledge base, the first knowledge base comprising annotated data relating problem elements to target elements;
receiving a medical data set;
determining a co-occurrence of data elements in at least a subset of the medical data set;
training a neural network model on the first knowledge base and the determined co-occurrence of data;
scoring, using the trained neural network model and the determined co-occurrence of the data elements, data relations in the first knowledge base; and
constructing, based on the scored data relations, a second knowledge base.

19. The one or more non-transitory, computer-readable media of claim 18, wherein the computer executable instructions, cause at least one processor to perform actions comprising:
determining embeddings from the medical data set; and
initializing the neural network model based on the embeddings.

20. The one or more non-transitory, computer-readable media of claim 18, wherein the computer executable instructions, cause at least one processor to perform actions comprising:
generating at least one list of patient problems and related medical elements using the second knowledge base;
receiving a patient medical record; and
reorganizing the patient medical record into a problem-oriented view using the at least one list.
